(19) United States
(12) Patent Application Publication
(10) Pub. No.: US 2025/0198949 A1
(43) Pub. Date: Jun. 19, 2025

(54) RADIO/MICROWAVE FREQUENCY GAS SENSOR

(71) Applicant: Know Labs, Inc., Seattle, WA (US)

(72) Inventors: Barry SHELTON, Seattle, WA (US); Dominic KLYVE, Seattle, WA (US)

(21) Appl. No.: 18/545,195

(22) Filed: Dec. 19, 2023

(51) Int. Cl. G01N 22/00; G01N 1/22; G01N 33/00; G08B 17/103

(52) U.S. Cl. CPC G01N 22/00; G01N 1/2247; G01N 33/0063; G08B 17/103

(57) ABSTRACT

A gas sensor that uses one or more radio or microwave frequency signals transmitted into a gaseous sample to aid in making a determination about the gaseous sample. This is generally referred to as characterizing a gaseous sample. Characterizing the gaseous sample can include, but is not limited to: detecting one or more gases in the sample; detecting the lack of one or more gases in the sample; determining the molecular makeup of homogenous or heterogeneous gases; determining the total makeup of a gaseous sample with or without suspended vapors or aerosols; determining the amount of suspended vapors or aerosols in a gaseous sample; determining whether the gaseous sample has an expected makeup; determining whether the gaseous sample deviates from an expected makeup; and others.

### FIELD

This disclosure relates generally to apparatus, systems and methods of characterizing a gas sample using a sensor that operates in the radio or microwave frequency range of the electromagnetic spectrum.

### BACKGROUND

A number of products exist that detect or otherwise characterize gases. For example, sensors are known that can detect concentrations of eddy covariance or eddy flux of carbon dioxide, oxygen, and methane in gaseous samples using waves in the infrared band of the electromagnetic spectrum. Simple smoke detectors use photo-electric sensor that operate in the optical band of the electromagnetic spectrum. Carbon monoxide detectors detect carbon monoxide via a chemical reaction that can require minutes or even hours to provide warning signals after carbon monoxide has reached levels dangerous to human health. Most modern carbon dioxide detectors use infrared light to detect carbon dioxide in a sample of air.

### SUMMARY

A gas sensor is described herein that uses signals in the radio frequency/microwave frequency range. The gas sensor and methods described herein use one or more radio or microwave frequency signals transmitted into a gaseous sample to aid in making a determination about the gaseous sample. This is generally referred to as characterizing a gaseous sample. Characterizing the gaseous sample can include, but is not limited to: detecting one or more gases in the sample; detecting the lack of one or more gases in the sample; determining the molecular makeup of homogenous or heterogeneous gases; determining the total makeup of a gaseous sample with or without suspended vapors or aerosols; determining the amount of suspended vapors or aerosols in a gaseous sample; determining whether the gaseous sample has an expected makeup; determining whether the gaseous sample deviates from an expected makeup; and others.

A gaseous sample (or gas sample) described herein is a sample that is primarily in gaseous form. The sample may or may not include suspended vapors or aerosols. Examples of vapors or aerosols include, but are not limited to, solids such as smoke particles, water/moisture particles, and other particles. The gaseous sample may be a homogenous gas or a heterogeneous gas.

A gas sensor described herein can include a transmit antenna, such as a strip of conductive material, that is configured to transmit a transmit signal that is in a radio or microwave frequency range of the electromagnetic spectrum into a gaseous sample. A transmit circuit is electrically connectable to the transmit antenna and is configured to generate the transmit signal to be transmitted by the transmit antenna. A receive antenna, such as a strip of conductive material, is located relative to the transmit antenna to detect a signal resulting from transmission of the transmit signal by the transmit antenna into the gaseous sample. A receive circuit is electrically connectable to the receive antenna where the receive circuit is configured to receive the signal detected by the receive antenna. The gaseous sample is located in a gaseous sample space adjacent to the transmit antenna and the receive antenna.

In operation, the signal detected by the receive antenna that results from transmitting the signal by the transmit antenna is compared with at least one sample gas signal to make a determination about the gaseous sample. The comparison can take many forms. For example, the comparison can be whether the detected signal matches the sample gas signal. For example, the comparison can be whether the detected signal deviates from the sample gas signal, for example by a predetermined amount.

A gas characterization method can include transmitting a transmit signal from a transmit antenna into a gaseous sample, where the transmit signal is in a radio or microwave frequency range of the electromagnetic spectrum. A response signal that results from transmission of the transmit signal into the gaseous sample is detected at a receive antenna. The detected response signal (or a derivative thereof) is then used to characterize the gaseous sample.

### DRAWINGS

FIG. 1 depicts a gas sensor as described herein.

FIG. 2 depicts a portion of another embodiment of a gas sensor.

FIG. 3 depicts a portion of still another embodiment of a gas sensor.

FIG. 4 depicts a signal storage described herein.

FIG. 5 depicts a gas characterization method described herein.

FIG. 6 depicts another embodiment of a gas sensor.

### DETAILED DESCRIPTION

The following is a detailed description of apparatus, systems and methods of characterizing a gaseous sample via spectroscopic techniques using frequencies in the radio or microwave frequency bands of the electromagnetic spectrum. A gas sensor described herein includes at least one antenna which functions as a transmit antenna to transmit an electromagnetic signal in the radio or microwave frequency range into a gaseous sample and at least one antenna which functions as a receive antenna that receives/detects an electromagnetic signal in the radio or microwave frequency range resulting from transmission of the electromagnetic signal into the gaseous sample. The signal(s) detected by the receive antenna(s) is then used to characterize the gaseous sample, for example by comparing the detected signal to one or more stored sample signals.

Based on the characterization, a warning/notification/alert signal can be generated and transmitted to a warning/notification/alert device. The warning/notification/alert signal can alert a user to a problem with the gaseous sample, and therefore of the surrounding environment. The warning/notification/alert signal can alert a user that the gaseous sample is as expected, and therefore the surrounding environment is acceptable. The warning/notification/alert signal can alert a user that the gaseous sample is trending over time toward a potential problem. The warning/notification/alert signal can alert a user that the gaseous sample has been stable over a time period. The warning/notification/alert device can generate a visual notice, such as a light or text on a display screen, an audible notice, a haptic notice. The results of the characterization can also be used to control an element, such as a valve, an exhaust fan, a door, or the like that can result in a modification of the environment in which the gas sensor is located.

Examples of sensors that can detect analytes using radio frequency or microwave frequency signals are described in U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, each of which is incorporated herein by reference in its entirety.

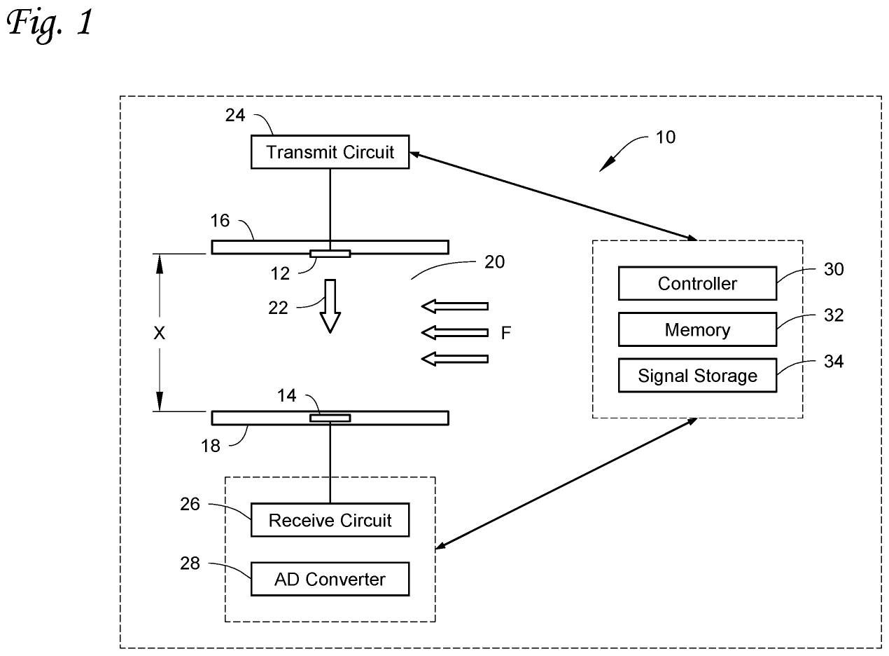

Referring to FIG. 1, an example of a gas sensor 10 is depicted. The gas sensor 10 includes a signal transmit side with at least one transmit antenna 12, and a receive side with at least one receive antenna 14. The transmit antenna 12 can be supported on a substrate 16 such as a plate. Similarly, the receive antenna 14 can be supported on a substrate 18 such as a plate. The antennas 12, 14 are spaced generally opposite from one another by a distance X to define a gaseous sample detection space 20 therebetween.

The transmit antenna 12 is configured to transmit one or more transmit signals 22 that is in a radio or microwave frequency band of the electromagnetic spectrum into a gaseous sample located in the detection space 20 and toward the receive antenna 14. The receive antenna 14 is configured to detect a signal that results from transmission of the transmit signal 22 into and through some or all of the gaseous sample. For example, the receive antenna 14 can detect a modified version of the transmit signal 22 after the transmit signal 22 passes through the gaseous sample. The transmit signal 22 may be altered by the gaseous sample and constituents thereof as it passes through the gaseous sample. The modified signal detected by the receive antenna 14 can then be used to make a determination about, i.e. characterize, the gaseous sample. The gaseous sample may be a multi-component gas such as air, or a single component (or substantially single component) gas such as a pure elemental gas like oxygen, nitrogen, etc.

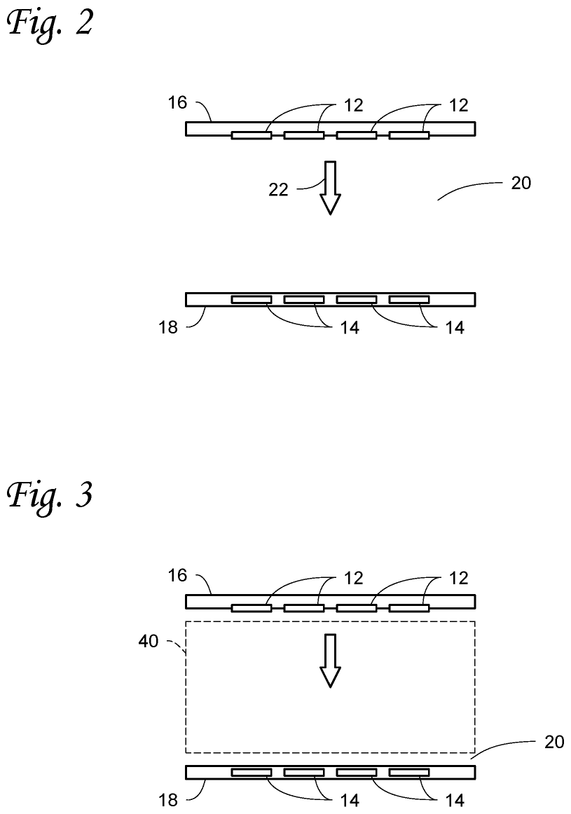

The gaseous sample in the detection space 20 may be from the ambient environment in which the gas sensor 10 is disposed, for example as illustrated in FIG. 2. In this embodiment, the gaseous sample is not constrained while in the detection space 20 and the gaseous sample may be generally stationary during detection except for any currents that may be present in the ambient environment. In another embodiment, the gaseous sample may be caused to flow through the detection space 20 during detection as indicated by the arrows F in FIG. 1. In another embodiment, the gaseous sample may be disposed within a container 40 (shown in dashed lines in FIG. 3) that is disposed between the transmit antenna 12 and the receive antenna 14 whereby the gaseous sample is substantially stationary during detection except for any currents that may be present in the container 40.

FIGS. 2 and 3 illustrate alternative embodiments of the gas sensor 10. In FIGS. 2 and 3, elements that are the same as or similar to elements in FIG. 1 are referenced using the same reference numerals. In FIGS. 2 and 3, the transmit side is depicted as including a plurality of the transmit antennas 12 on the substrate 16, and the receive side is depicted as including a plurality of the receive antennas 14 on the substrate 18. Any one or more of the transmit antennas 12 can be controlled to transmit the transmit signal(s) 22, and any one or more of the receive antennas 14 can be controlled to detect the signal(s) transmitted through the gas sample. Controlling any of a number of transmit antennas to transmit an RF signal and any of a number of receive antennas to receive an RF signal is disclosed in U.S. Pat. Nos. 11,058,331, 11,193,923, and 11,330,997 each one of which is incorporated herein by reference in its entirety.

Returning to FIG. 1, the transmit antenna 12 can have any form suitable for transmitting the RF/microwave transmit signal 22. For example, the transmit antenna 12 can comprise a strip or patch of conductive material such as metal or other material that can transmit signals in the radio or microwave frequency range of the electromagnetic spectrum. The transmit antenna 12 may reside on the surface of the plate 16, the transmit antenna 12 may partially protrude from (i.e. be partially embedded within) the plate 16 as depicted in FIG. 1, or the transmit antenna 12 may be completely embedded within the plate 16. The plate 16 may be formed in a manner so it does not interfere with operation of the transmit antenna 12, for example being formed of a non-conductive material such as plastic.

The receive antenna 14 can have any form suitable for receiving RF/microwave signals after passing through the gaseous sample. For example, the receive antenna 14 can comprise a strip or patch of conductive material such as metal or other material that can receive signals in the radio or microwave frequency range of the electromagnetic spectrum. The receive antenna 14 may reside on the surface of the plate 18, the receive antenna 14 may partially protrude from (i.e. be partially embedded within) the plate 18, or the receive antenna 14 may be completely embedded within the plate 18 as depicted in FIG. 1. The plate 18 may be formed in a manner so it does not interfere with operation of the receive antenna 14, for example being formed of a non-conductive material such as plastic.

One or both of the antennas 12, 14 may be position adjustable in order to alter the distance X between the antennas 12, 14. For example, one or both of the plates 16, 18 can be mounted so as to be positon adjustable to change the distance X. Alternatively, the antennas 12, 14 may be position adjustable while keeping the plates 16, 18 stationary. Changing the distance X changes the sensing performance of the gas sensor 10. In one embodiment, the distance X may be no greater than 2.0 feet and at least 1.0 mm. The distance X may be at least 1.0 mm and less than or equal to 1.0 foot. Long range communication systems that utilize a transmit antenna and a receive antenna use signal modulation (either frequency or amplitude modulation) to communicate. In contrast, in an embodiment, the antennas 12, 14 described herein do not use modulated signals at any given frequency. Instead, the techniques described herein determine signal loss caused by transmitting the signal into the gaseous sample to characterize the gaseous sample. In another embodiment, the angles and/or shapes of the antennas 12, 14 may be changed in order to alter the performance of the sensor. An example of an RF analyte sensor with adjustable transmit and/or receive components is disclosed in U.S. Pat. No. 11,696,698, the entire contents of which are incorporated by reference.

Referring to FIG. 6, in another embodiment, the transmit antenna(s) 12 and the receive antenna(s) 14 can be located on the same side of the gaseous sample. In this embodiment, the transmit antenna(s) 12 transmits the signal 22 into the gaseous sample, and the receive antenna(s) 14 detects a return signal 23 that results from the transmitted signal 22. Similar to the embodiments described above, the gaseous sample in the detection space 20 may be from the ambient environment in which the gas sensor is disposed, like illustrated in FIG. 2, where the gaseous sample is not constrained while in the detection space 20 and the gaseous sample may be generally stationary during detection except for any currents that may be present in the ambient environment. Alternatively, the gaseous sample may be caused to flow through the detection space 20 during detection like depicted in FIG. 1. In another embodiment, the gaseous sample may be disposed within a container, like shown in FIG. 3.

Returning to FIG. 1, the transmit signal 22 is generated by a transmit circuit 24 that may be connectable to the transmit antenna 12. A receive circuit 26 is connectable to the receive antenna 14 to convert the electromagnetic energy detected by the receive antenna 14 into one or more signals reflective of the response detected by the receive antenna 14. An analog to digital (AD) converter 28 may be provided to convert the signal detected by the receive circuit 26 from an analog signal into a digital processor readable format. The AD converter 28 may be separate from the receive circuit 26 or the AD converter 28 may be considered part of the receive circuit 26. Similarly, a digital to analog converter, which may be part of or separate from the transmit circuit 24, converts a digital signal into an analog signal for transmission by the transmit antenna 12.

A controller 30 controls operation of the gas sensor 10, including controlling the transmit circuit 24 and the receive circuit 26. The controller 30 may include a processor, such as a micro-controller unit (MCU), which may facilitate the operation of the gas sensor 10 according to instructions stored in memory 32. The processor may include suitable logic, circuitry, interfaces, and/or code that may be configured to execute a set of instructions stored in the memory 32. The processor may be a hardware component that performs arithmetic, logic, and control operations on data. The processor may be comprised of the arithmetic logic unit, control unit, memory subsystems, and other subsystems. The processor may be responsible for performing signal comparison, as well as arithmetic and logical operations on data. The processor may include components for addition, subtraction, multiplication, and division and logical operations such as AND, OR, and NOT. The processor may be responsible for fetching instructions from the memory 32, decoding them, and executing them. The processor may manage the flow of data between different components of the gas sensor as a whole, ensuring that operations are performed in the correct order, and that data is transferred efficiently. The processor may provide fast access to frequently used data and instructions. The processor may include components such as caches, registers, and pipelines, which are designed to minimize the time required to access and manipulate data. The processor may include various other components and subsystems, such as instruction set architecture (ISA), which may define the set of instructions that the processor can execute. The processor may specify the format of instructions and data, the addressing modes used to access memory 32 and I/O devices, and the interrupt and exception handling mechanisms used to manage errors and other events. The processor may include advanced instruction execution capabilities, support for virtualization and parallel processing, and power management mechanisms that reduce energy consumption and heat dissipation. In some embodiments, the controller 30 may be a programmable logic controller (PLCs), or digital signal processors (DSPs) and may be implemented as software components running on general-purpose computers or embedded systems.

A signal storage 34 stores one or more signals. The signal storage 34 may be any form of storage that is suitable for storing signal waveforms for use in comparing, in real-time, to the signal detected by the receive antenna 14 to characterize the gaseous sample. For example, the signal storage 34 may be a database. The signal storage 34 may be part of the memory 32 or may be at a location separate from the memory 32. The signal waveform comparison may be performed by the controller 30. The techniques for comparison of signal waveforms are well-known in the art.

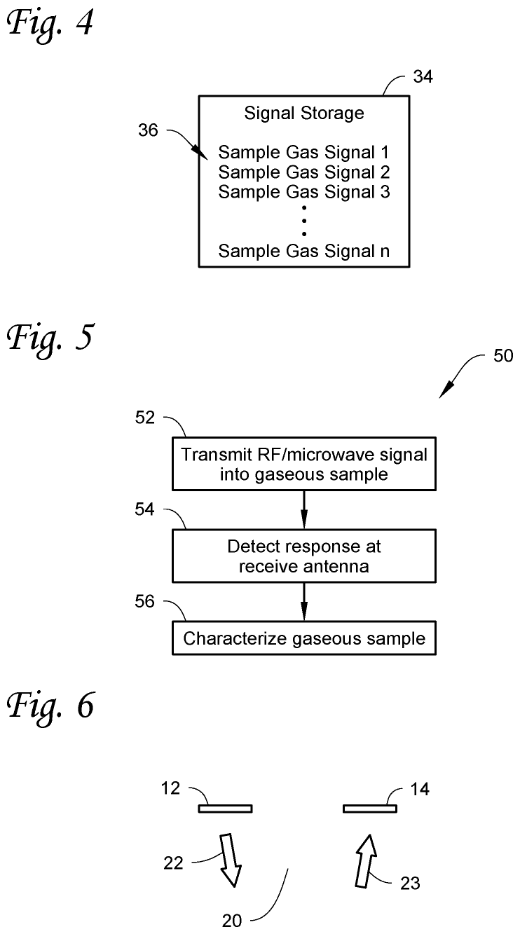

FIG. 4 depicts an example of the signal storage 34 storing a plurality of sample gas signals 36. The signal storage 34 may store a single one of the signals 36. The signals 36 that are stored can be based on the purpose of the comparison. Many signals 36 are possible. Each signal 36 can represent a different level of a particular gas, such as carbon monoxide, carbon dioxide, etc., in the gaseous sample. Each signal 36 can represent a different level of a particular gas, such as carbon monoxide, carbon dioxide, etc., in the gaseous sample. Each signal 36 may represent a different molecular makeup of homogenous or heterogeneous gas samples. A homogenous gas is a gas mixture in which the components mix with each other and its composition is uniform throughout the solution. A heterogeneous gas is a gas mixture in which the composition is not uniform throughout and different components are observed. Each signal 36 may represent a different amount of suspended vapors or aerosols in gas samples. Different signal sets may be stored. For example, one set of stored signals 36 can be provided for one type of gas in the gaseous sample, and a second set of stored signals 36 can be provided for a second type of gas in the gaseous sample.

The signal waveform comparison may be to match the detected signal waveform with the stored signal(s) 36. This may assist in detecting a particular gas in the gaseous sample; detecting a lack of a gas in the gaseous sample; determining the molecular makeup of homogenous or heterogeneous gases; determining the total makeup of a gaseous sample with or without suspended vapors or aerosols; determining the amount of suspended vapors or aerosols in a gaseous sample; determining whether the gaseous sample has an expected makeup; etc. The signal comparison may be to determine if the detected signal waveform deviates from the stored signal 36 by a predetermined amount. For example, if the detected signal waveform deviates by 10% or more (or 3% or more; or 5% or more; etc.) from the stored signal 36, that can indicate a problem with the gaseous sample such as, but not limited to, an excessive amount of carbon monoxide in the gaseous sample, an excessive amount of carbon dioxide in the gaseous sample, an excessive amount of smoke (i.e. some particles) in the gaseous sample, and others.

Returning to FIG. 1, the controller 30 may be part of the transmit circuit 24, part of the receive circuit 26, or the transmit circuit 24 may include a controller that is separate from a controller included in the receive circuit 26. The gas sensor 10 can also include a power supply, such as a battery (not shown) which may or may not be rechargeable. In some embodiments, power can be provided from mains power, for example by plugging the gas sensor 10 into a wall socket via a cord connected to the gas sensor 10, or by another external power source. Further information on a transmit circuit and a receive circuit in an analyte sensor is disclosed in Pat. No. 11,063,373 the entire contents of which are incorporated herein by reference.

Referring to FIG. 5, an example of a gas characterization method 50, for example using the sensor 10 described herein, is illustrated. Further steps not illustrated in FIG. 5 may be included. In a step 52, a transmit signal that is in a radio or microwave frequency range of the electromagnetic spectrum is transmitted from the transmit antenna into the gaseous sample. The transmit signal may be a single discrete signal or the signal may be part of a frequency sweep in which a plurality of signals are transmitted. The frequency sweep may start at an initial frequency and end at a final frequency, with the signals separated by equal or unequal frequency steps. An example of performing frequency sweeps in a radio/microwave frequency sensor is disclosed in U.S. Pat. No. 11,033,208, the entire contents of which are incorporated herein by reference.

In a step 54, a response is detected at the receive antenna. The response results from transmission of the transmit signal into the gaseous sample. The transmitted signal may be altered by the gaseous sample and constituents thereof as it passes through the gaseous sample. The altered signal is detected and is used to characterize the gaseous sample.

At step 56, the detected response is then used to characterize the gaseous sample. For example, the detected signal waveform may, in its original form or after being converted to a digital waveform by the AD converter 28, be compared to one or more stored sample gas signals as described above for FIG. 4.

In one embodiment, the detected signal waveform may be compared to a single sample gas signal. If the detected signal waveform deviates from the sample gas signal, that can indicate a problem or deviates from an expected makeup, and a notification/alarm signal can be sent, for example to an alarm, to notify a user of a problem. The detected signal may need to deviate from the stored signal by a predetermined amount, such as 10% or more, 5% or more, 3% or more, or other quantifiable amount in order to generate a notification signal. The deviation of the detected signal from the stored signal can indicate a number of problems such as an excessive amount of carbon monoxide in the gaseous sample, an excessive amount of carbon dioxide in the gaseous sample, an excessive amount of smoke (i.e. some particles) in the gaseous sample, not enough oxygen or other gas in the gaseous sample, and others. The detected signal waveform may also match the sample gas signal which can indicate that the gaseous sample has an expected makeup.

In another embodiment, the detected signal waveform may be compared to a plurality of the sample gas signals in an effort to look for a match between the detected signal and one of the sample gas signals. A match can indicate that the gaseous sample is acceptable (i.e. the sample gas signals indicate acceptable gaseous samples), or a match can indicate that the gaseous sample is not acceptable (i.e. the sample gas signals indicate unacceptable gaseous samples).

The gas sensors 10 described herein may form or be incorporated into residential, industrial and/or commercial sensors/detectors. Examples of sensors that the gas sensor 10 can be incorporated into include, but are not limited to, a smoke detector, a carbon monoxide detector, a carbon dioxide detector, a methane detector, a toxic sensor, an LEL sensor, a hydrogen sulfide sensor, a sulfur dioxide sensor, combinations thereof and many others.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A gas characterization method, comprising:
transmitting a transmit signal from a transmit antenna into a gaseous sample, the transmit signal is in a radio or microwave frequency range of the electromagnetic spectrum;
detecting a response at a receive antenna that results from transmission of the transmit signal into the gaseous sample;
using the detected response to characterize the gaseous sample.

2. The gas characterization method of claim 1, wherein the gaseous sample is flowing.

3. The gas characterization method of claim 1, wherein the gaseous sample is disposed within a container.

4. The gas characterization method of claim 1, wherein the gaseous sample is a homogenous gas or a heterogeneous gas.

5. The gas characterization method of claim 1, wherein the gaseous sample comprises air.

6. The gas characterization method of claim 1, wherein the gaseous sample comprises carbon monoxide, and the transmit antenna and the receive antenna are in a carbon monoxide detector.

7. The gas characterization method of claim 1, wherein the gaseous sample comprises carbon dioxide, and the transmit antenna and the receive antenna are in a carbon dioxide detector.

8. The gas characterization method of claim 1, wherein the transmit antenna and the receive antenna are in a smoke detector.

9. The gas characterization method of claim 1, wherein characterizing the gaseous sample comprises comparing the detected response with a stored signal.

10. The gas characterization method of claim 1, wherein characterizing the gaseous sample comprises determining presence of a gas in the gaseous sample or determining absence of the gas in the gaseous sample.

11. The gas characterization method of claim 1, wherein characterizing the gaseous sample comprises determining whether the detected response matches a stored signal or determining whether the detected response deviates from a stored signal.

12. A gas sensor, comprising:
a transmit antenna that is configured to transmit a transmit signal, the transmit antenna comprises a strip of conductive material;
a transmit circuit that is electrically connectable to the transmit antenna, the transmit circuit is configured to generate the transmit signal to be transmitted by the transmit antenna, the transmit signal is in a radio or microwave frequency range of the electromagnetic spectrum;
a receive antenna that comprises a strip of conductive material that detects a signal resulting from transmission of the transmit signal by the transmit antenna;
a receive circuit that is electrically connectable to the receive antenna, the receive circuit is configured to receive the signal detected by the receive antenna;
a gaseous sample space adjacent to the transmit antenna and the receive antenna, the gaseous sample space having a gaseous sample into which the transmit signal is transmitted;
a data storage device that stores at least one sample gas signal;
a processor in communication with the receive circuit and with the data storage device, the processor is configured to execute instructions to compare the signal detected by the receive antenna with the at least one sample gas signal.

13. The gas sensor of claim 12, wherein the data storage device stores a plurality of sample gas signals.

14. The gas sensor of claim 12, wherein the data storage device comprises a database.

15. The gas sensor of claim 12, wherein the receive circuit includes an analog to digital converter that converts the signal detected by the receive antenna into a digital form.

16. A carbon monoxide detector that includes the gas sensor of claim 12.

17. A carbon dioxide detector that includes the gas sensor of claim 12.

18. A smoke detector that includes the gas sensor of claim 12.
